(19) United States
(12) Patent Application Publication
(10) Pub. No.: US 2014/0249156 A1
(43) Pub. Date: Sep. 4, 2014

(54) Treatment Of Blood Lipid Abnormalities And Other Conditions

(71) Applicant: GlaxoSmithKline LLC, Phidadelphia, PA (US)

(72) Inventors: Kathleen Brown, Research Triangle Park, NC (US); Andrew James Carpenter, Research Triangle Park, NC (US); Derek J.R. Nunez, Research Triangle Park, NC (US); Andrew Young, Research Triangle Park, NC (US)

(73) Assignee: GlaxoSmithKline LLC, Philadelphia, PA (US)

(21) Appl. No.: 14/353,147

(22) PCT Filed: Oct. 31, 2012
(86) PCT No.: PCT/US2012/062647

§ 371 Date: Apr. 21, 2014

Related U.S. Application Data
(60) Provisional application No. 61/558,588, filed on Nov. 11, 2011.

(51) Int. Cl. A61K 31/519; A61K 31/501; A61K 31/506; A61K 31/497; A61K 31/454

(52) U.S. Cl. CPC A61K 31/519; A61K 31/497; A61K 31/454; A61K 31/506; A61K 31/501
USPC 514/252.02; 514/262.1; 514/326; 514/256; 514/269; 514/265.1; 514/255.05

(57) ABSTRACT

There is provided a method of treatment of a patient suffering from blood lipid abnormality comprises the administration of a GPR119 agonist.

### FIELD OF THE INVENTION

The present invention relates to a method for treating a disease or condition in a mammal, which method comprises administering a GPR119 agonist to a mammal in need thereof.

### BACKGROUND OF THE INVENTION

GPR119 is a G-protein coupled receptor (GPCR). GPR119 is also referred to as IC-GPCR2 and also as RUP3. The expression profile of GPR119 indicates its potential utility as a target for the treatment of obesity and diabetes.

Patent application WO 2005/007658 (Jones et. al) discloses certain RUP3 modulators said to be useful in the prophylaxis or treatment of metabolic disorders such as diabetes and obesity.

Patent application WO 2008/081204 (Fyfe et. al) discloses certain agonists of GPR119 said to be useful for the treatment of obesity and diabetes.

Patent application WO 2008/083238 (Chen et. al) discloses certain IC-GPCR2 agonists said to be useful for the treatment of diabetes.

Patent application WO 2010/014593 (Carpenter et. al) discloses certain GPR119 agonists said to be useful in the treatment and prevention of metabolic disorders, including diabetes and obesity.

Patent application WO 2007/035355 (Jones et. al) discloses a RUP3 modulating compound, said to be useful in the treatment of diabetes and obesity.

Patent application WO 2008/008887 discloses certain GPR119 agonists said to be useful in the treatment and prevention of metabolic disorders, including diabetes and obesity.

Patent application WO 2007/116229 (Bertram et. al) discloses certain GPR119 agonists said to be useful in the treatment and prevention of metabolic disorders, including diabetes and obesity.

Patent application WO 2008/070692 (Fang et. al) discloses certain GPR119 agonists including the compound of example 100, 5-[({1-[3-(1-methylethyl)-1,2,4-oxadiazol-5-yl]-4-piperidinyl}methyl)oxy]-2-[4-(methylsulfonyl)phenyl]pyridine.

### SUMMARY OF THE INVENTION

The present invention provides a method for treating a mammal suffering from an abnormality of blood lipids wherein said method comprises administering a GPR119 agonist, other than 5-[({1-[3-(1-methylethyl)-1,2,4-oxadiazol-5-yl]-4-piperidinyl}methyl)oxy]-2-[4-(methylsulfonyl)phenyl]pyridine or a pharmaceutically acceptable salt thereof, to a mammal in need thereof. An example of a blood lipid abnormality is dyslipidemia.

### BRIEF DESCRIPTION OF THE DRAWINGS

FIG. 1. The appearance of ¹⁴C cholesterol (CPM) as a function of time in the serum of rats which received a single dose of either vehicle or Compound A (30 mg/kg).

FIG. 2. Comparison of two GPR119 agonists. The appearance of ¹⁴C cholesterol (CPM) as a function of time in the serum of rats which received a single dose of either vehicle or a GPR119 agonist (Compound A at 3 or 10 mg/kg or Compound B at 30 mg/kg.

FIG. 3. Comparison of two GPR119 agonists. The appearance of ³H cholesterol (CPM) as a function of time in the serum of rats which received a single dose of either vehicle or a GPR119 agonist (Compound A at 3 or 10 mg/kg or Compound B at 30 mg/kg).The compounds are the same as in FIG. 2 but the cholesterol is labeled with ³H rather than ¹⁴C.

FIG. 4. Comparison of four GPR119 agonists from published literature. The appearance of ³H cholesterol (CPM) as a function of time in the serum of rats which received a single dose of either vehicle or a diverse set of GPR119 agonists (compounds C-F at 30 mg/kg).

FIG. 5. Comparison of two GPR119 agonists from patents or published literature. The appearance of ³H cholesterol (CPM) as a function of time in the serum of rats which received a single dose of either vehicle or a diverse set of GPR119 agonists (compounds G and H at 30 mg/kg).

FIG. 6. Comparison of two GPR119 agonists from patents or published literature. The appearance of ³H cholesterol (CPM) as a function of time in the serum of rats which received a single dose of either vehicle or a diverse set of GPR119 agonists (compounds I and J at 30 mg/kg)

### DETAILED DESCRIPTION OF THE INVENTION

There is provided in the present invention a method for treating a mammal, preferably a human, suffering from an abnormality of blood lipids, wherein said method comprises administering a GPR119 agonist to a mammal in need thereof and wherein said GPR119 agonist is other than 5-[({1-[3-(1-methylethyl)-1,2,4-oxadiazol-5-yl]-4-piperidinyl}methyl)oxy]-2-[4-(methylsulfonyl)phenyl]pyridine (herein after referred to as “Compound A”) or a pharmaceutically acceptable salt thereof.

Patent application U.S. Ser. No. 61/349,268 (and PCT/US2011/037912) discloses a method for treating a human suffering from an abnormality of blood lipids wherein said method comprises administering a pharmaceutical composition comprising Compound A or a pharmaceutically acceptable salt thereof to a human in need thereof. An example of a blood lipid abnormality is dyslipidemia. Another example is atherosclerotic cardiovascular disease. See also Example 100 in WO 2008/070692 for a preparation of Compound A. Patent application U.S. Ser. No. 61/349,268 (and PCT/US2011/037912) disclose that the administration of Compound A or a pharmaceutically acceptable salt thereof has the following effects on abnormal blood abnormalities in humans: increasing fasting HDL cholesterol, reduced fasting LDL cholesterol, reducing fasting non-HDL cholesterol, reducing fasting total cholesterol, reducing fasting total cholesterol:HDL cholesterol ratio, reducing fasting LDL cholesterol:HDL cholesterol ratio, reducing fasting and prandial triglycerides, reducing fasting ApoB/B100, reducing fasting ApoB/B100:ApoA1 ratio, reducing fasting ApoE, reducing fasting plasma alanine aminotransferase, and reducing fasting plasma uric acid. This patent application claims the use of the above compound to treat atherosclerotic cardiovascular disease, non-alcoholic fatty liver disease, chronic hepatitis C virus infection, and hyperuricemia.

The present invention demonstrates that the effects noted in the previous patent application may be generally applicable to all GPR119 agonists. In particular, the present application demonstrates that GPR119 agonists of a variety of structure classes impede the appearance of cholesterol in circulation. The GPR119 agonists tested are as follows: Compound A; Compound B (2-[((1S)-1-{1-[3-(1-methylethyl)-1,2,4-oxadiazol-5-yl]-4-piperidinyl}ethyl)oxy]-5-[4-(methylsulfonyl)phenyl]pyrazine) is the compound of Example 158 in patent application WO 2008/070692; Compound C (1-methylethyl 4-({1-[2-fluoro-4-(methylsulfonyl)phenyl]-1H-pyrazolo[3,4-d]pyrimidin-4-yl}oxy)-1-piperidinecarboxylate) is Compound A129 in patent application WO 2005/007658; Compound D (4-((1R)-3-{[3-fluoro-4-(methylsulfonyl)phenyl]oxy}-1-methylpropyl)-1-[3-(1-methylethyl)-1,2,4-oxadiazol-5-yl]piperidine) is the compound of Example 7 in patent application WO 2008/081204; Compound E (5-ethyl-2-{4-[4-({[4-(1H-tetrazol-1-yl)phenyl]oxy}methyl)-1,3-thiazol-2-yl]-1-piperidinyl}pyrimidine) is the compound of Example 52 in patent application WO 2008/083238; Compound F (4-{5-[((1S)-1-{1-[3-(1-methylethyl)-1,2,4-oxadiazol-5-yl]-4-piperidinyl}ethyl)oxy]-2-pyrazinyl}pyridazine) is the compound of Example 2 in patent application WO 2010/014593; Compound G (1-methylethyl 4-({5-methyl-6-[(2-methyl-3-pyridinyl)oxy]-4-pyrimidinyl}oxy)-1-piperidinecarboxylate hydrochloride) is the compound of Formula I in patent application WO 2007/035355; Compound H (1-methylethyl 4-({7-[2-methyl-4-(methylsulfonyl)phenyl]-6,7-dihydro-5H-pyrrolo[2,3-d]pyrimidin-4-yl}oxy)-1-piperidinecarboxylate) was not specifically exemplified but is generically encompassed by patent application WO 2008/008887; Compound I (2-[((1S)-1-{1-[2-(1-methylethyl)-2H-tetrazol-5-yl]-4-piperidinyl}ethyl)oxy]-5-[4-(methylsulfonyl)phenyl]pyrazine) is Compound 191 in patent application WO 2008/070692; Compound J (1,1-dimethylethyl 4-[((1R)-1-{3-[3-fluoro-4-(methylsulfonyl)phenyl]-1,2,4-oxadiazol-5-yl}ethyl)oxy]-1-piperidinecarboxylate) is the compound of Example 24 in patent application WO 2007/116229. Each of these compounds can be prepared, for example, as described in the referenced patent application.

In the method of treatment herein, a compound of the method may be administered in the form of a pharmaceutical composition or formulation. Such a pharmaceutical composition may employ one or more pharmaceutically acceptable carriers, excipients, or diluents. The carrier(s), diluents(s), and excipient(s) must be acceptable in the sense of being compatible with the other ingredients of the formulation and not deleterious to the recipient of the pharmaceutical composition.

A compound of the method of this invention may be employed alone or in combination with other therapeutic agents. The compounds of this invention may be administered together or separately and, when administered separately, administration may occur simultaneously or sequentially, in any order. The amount of the compound of the invention and the other pharmaceutically active agent(s) and the relative timings of administration will be selected in order to achieve the desired combined therapeutic effect. The administration of the combination may be concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. Such current therapeutic agents can include, but are not limited to, statins (3-hydroxy-3-methylglutaryl-coenzyme (HMG-CoA) reductase inhibitors), ezetimibe, fibrates (peroxisome proliferator activated receptor alpha agonists) including, but not limited to, fenofibric acid, bezafibrate, gemfibrozil, niacin/nicotinic acid, intestinal bile acid sequestrants, intestinal lipase inhibitors, interferons, ribavirin, allupurinol, corticosteroids, non-steriodal anti-inflammatory drugs, acetaminophen, febuxostat, colchicine, probenecid, metformin, thiazolidinediones, and/or vitamin E.

The invention may be further described by the experiments which follow.

### Experimental

Rationale:

Agents which impede the appearance of cholesterol in circulation via altering intestinal absorption provide, in addition to statins, an additional avenue for control of hypercholesterolemia and reduction in cardiovascular morbidity and mortality. It was previously discovered that Compound A lowers total and LDL cholesterol and raises HDL cholesterol. Additionally, the rise in serum triglycerides that occurs following meals was significantly blunted in patients treated with Compound A; therefore, it is considered that activation of the GPR119 receptor in the gastrointestinal tract is a mechanism whereby these beneficial effects may be mediated.

To determine whether the observed (with Compound A) GPR119 mediated effects on serum cholesterol in humans could be recapitulated in an animal model, Compound A and other GPR119 agonists were tested.

A rat model (male Crl:CD(SD) rats) was used for the evaluation of a diverse set of GPR119 agonists. The objective of the studies was to determine if administration of a single dose of an agonist affects the appearance of chylomicrons containing either ³H- or ¹⁴C-cholesterol in circulation of fasted Crl:CD(SD) rats.

Methods:

In order to track chylomicron appearance in circulation, radiolabeled cholesterol (with or without radiolabeled retinol) was co-administered with olive oil by oral gavage. Both tracers label the chylomicron core as the cholesterol administered is esterified in the enterocyte, packaged into the chylomicron, and released into the lymphatic system for transport to the systemic circulation. In order to assess appearance and accumulation of the newly formed chylomicrons in circulation, triton was administered by intravenous injection to block the clearance of these particles from circulation.

The rats had been previously instrumented with an implanted jugular catheter to facilitate obtaining multiple blood samples without stress to the animal. Rats were fasted overnight prior to labeled oral fat challenge. All compounds were dosed one hour prior to the challenge test. Treatment groups were randomized by body weight.

All compounds were administered at 5 mL/kg by the oral route as suspensions in hydroxypropylmethylcellulose (0.5%) which contained 0.1% Tween 80. Triton (250 mg/kg) was administered by intravenous injection immediately prior to an oral gavage of the tracer mixed with olive oil. Total volume of olive oil administered per rat was 2 mL, which contained (per 2 mL olive oil) either:

5-10 uCi ¹⁴C-cholesterol (alone or in combination with 80 uCi ³H-retinol)

5-20 uCi ³H-cholesterol.

Blood samples were taken at the following time points: 0.25, 0.5, 0.75, 1, 2, 3, 4, 5 and 6 hours post olive oil gavage. Samples were spun at 8,000×g for 8 minutes at room temperature and 100 uL aliquots of serum counted for ³H or ¹⁴C.

Results:

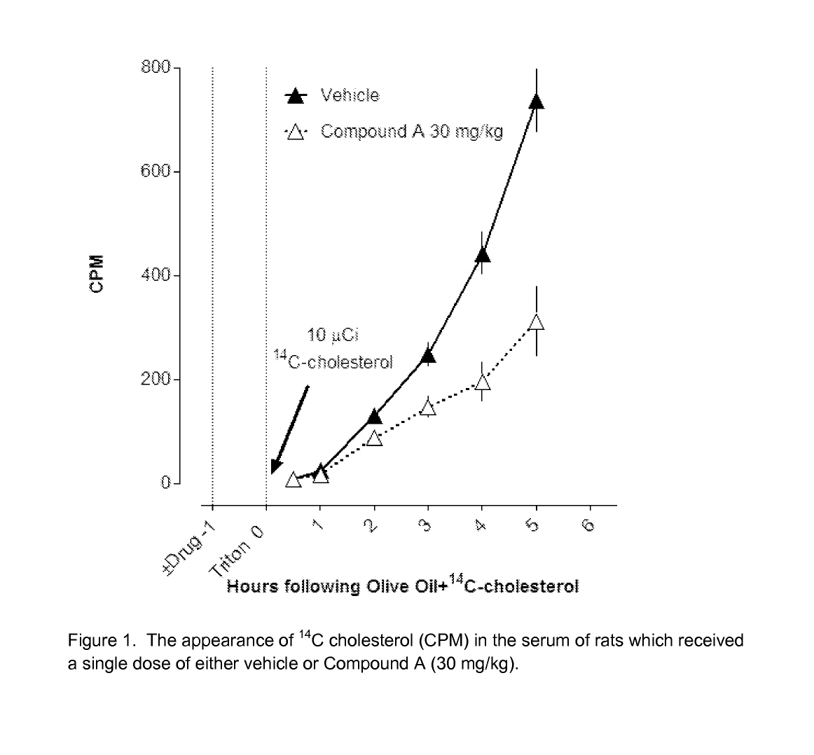

A demonstrated in FIG. 1, pretreatment with Compound A (30 mg/kg) significantly reduced the appearance of ¹⁴C labeled chylomicrons in circulation after rats received olive oil containing ¹⁴C cholesterol compared to vehicle treated rats.

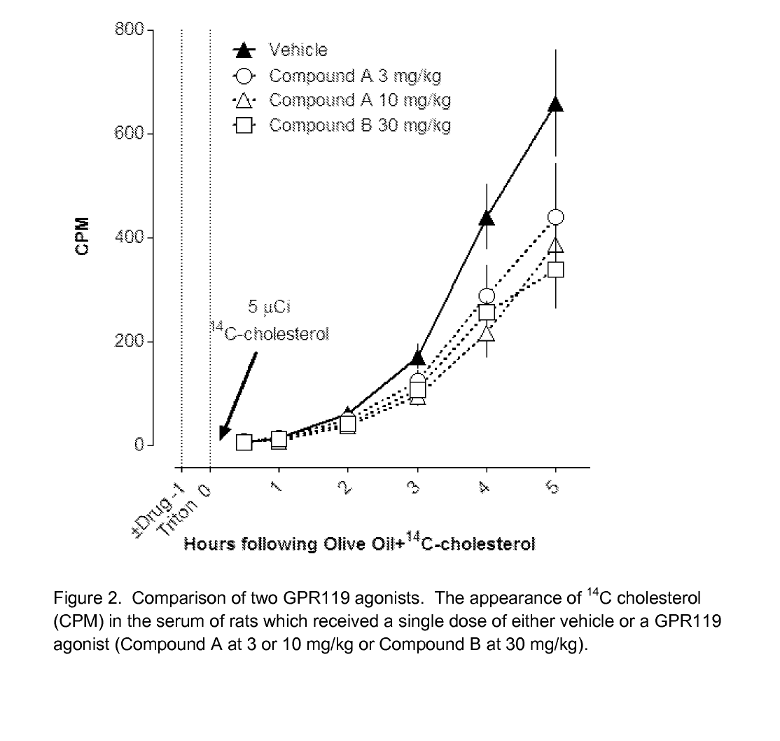

As demonstrated in FIG. 2, pretreatment with GPR119 agonists (Compound A at 3 or 10 mg/kg or Compound B at 30 mg/kg) significantly reduced the appearance of ¹⁴C labeled chylomicrons in circulation after rats received olive oil containing ¹⁴C cholesterol compared to vehicle treated rats.

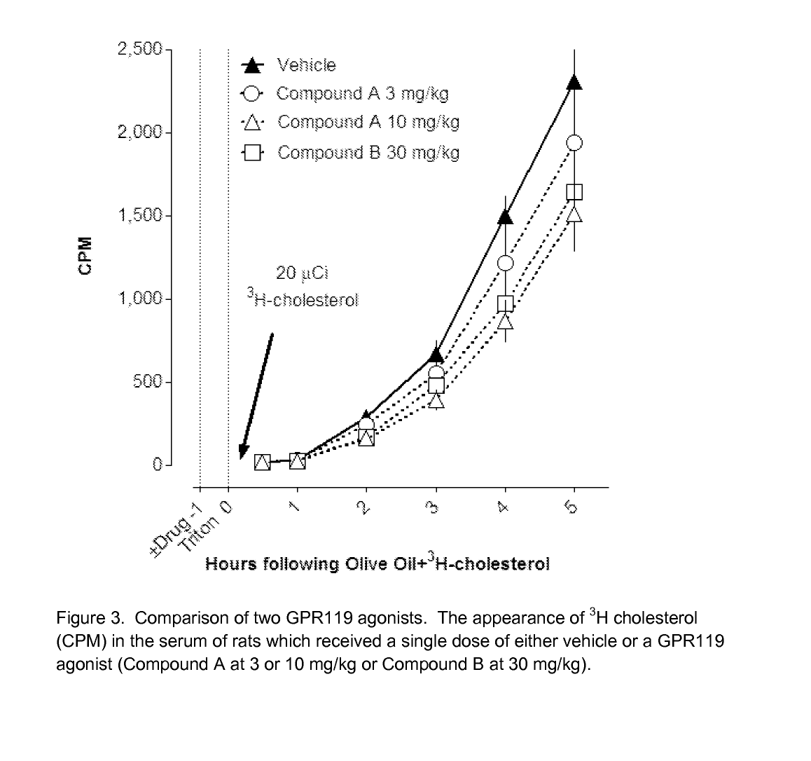

The data shown in FIG. 3 is a comparison of the same compounds as in FIG. 2 with a different tracer label on the cholesterol. Pretreatment with either agonist decreased the appearance of ³H cholesterol (CPM) in the serum of rats which received a single dose of either vehicle or a GPR119 agonist (Compound A at 3 or 10 mg/kg or Compound B at 30 mg/kg).

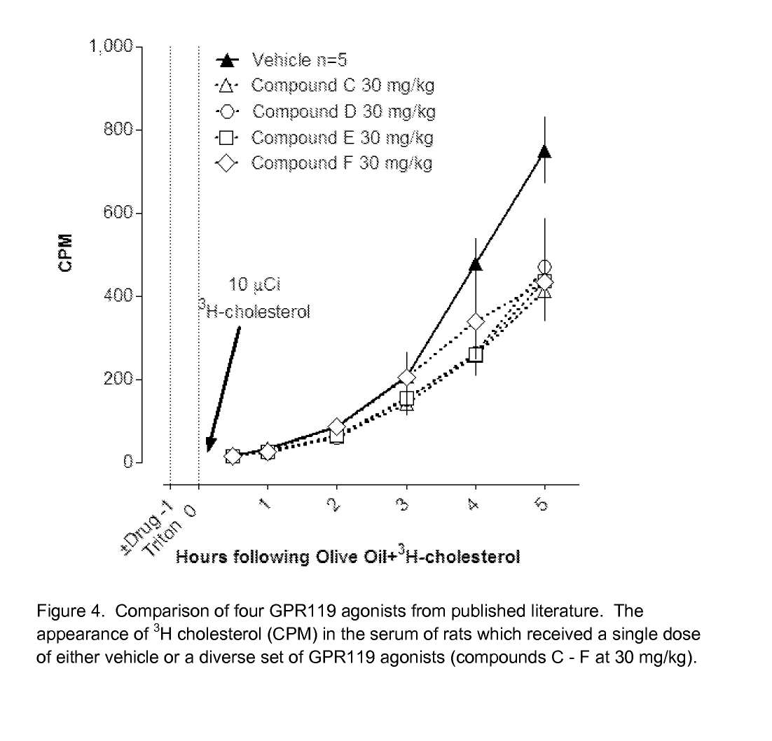

Data from a diverse set of compounds from published literature are shown in FIG. 4. The appearance of ³H cholesterol (CPM) in the serum of rats which received a single dose of either vehicle or GPR119 agonists labeled as compounds C-F each dosed at 30 mg/kg. Pretreatment with all agonists significantly reduced the appearance of ³H labeled chylomicrons in circulation after rats received olive oil containing ¹³H cholesterol.

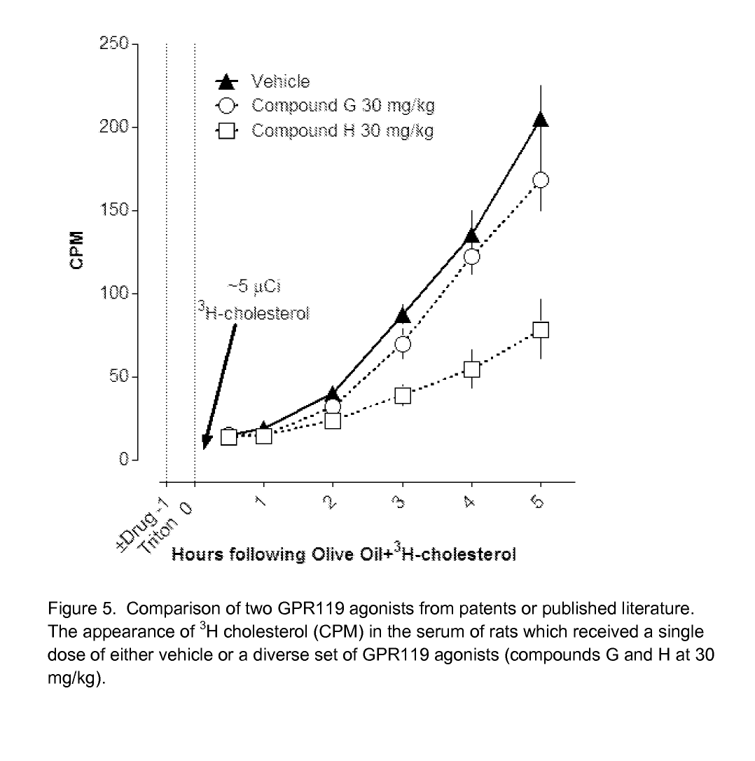

Comparison between two GPR119 agonists from patents or published literature is shown in FIG. 5. The appearance of ³H cholesterol (CPM) in the serum of rats was significant reduced after a single dose of either vehicle or GPR119 agonist compounds G and H at 30 mg/kg.

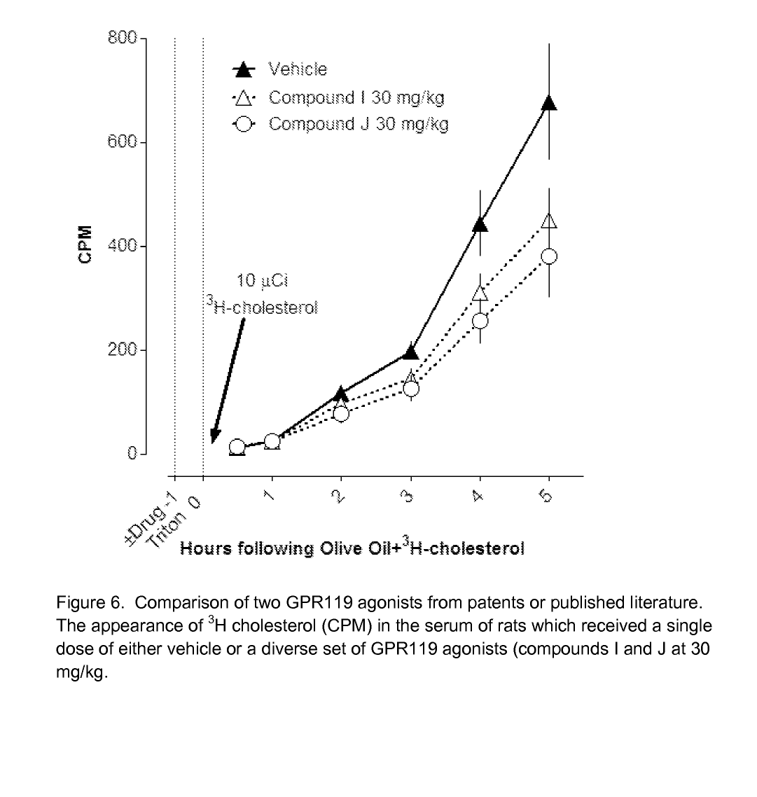

Similarly FIG. 6 shows data in which the appearance of ³H cholesterol (CPM) in the serum of rats was significant reduced after a single dose of either vehicle or GPR119 agonist compounds I and J at 30 mg/kg.

The Data show that all of the tested GPR119 agonists impede the appearance of labeled cholesterol in circulation. Applicants believe that this shows that this effect is a GPR119 mediated effect and should be generally seen with any GPR119 agonist and therefore, GPR119 agonists in general should be useful for treating dyslipidemia.

What is claimed is:

1. A method for treating an abnormality of blood lipids in a mammal, wherein said method comprises administering a GPR119 agonist to a mammal in need thereof, and wherein said GPR119 agonist is other than 5-[({1-[3-(1-methylethyl)-1,2,4-oxadiazol-5-yl]-4-piperidinyl}methyl)oxy]-2-[4-(methylsulfonyl)phenyl]pyridine or a pharmaceutically acceptable salt thereof.

2. The method of claim 1 wherein the abnormality of blood lipids is dyslipidemia.

3. The method of claim 1 wherein the abnormality of blood lipids is atherosclerotic cardiovascular disease.

4. The method of claim 1 wherein said mammal is a human.

5. The method of claim 4 wherein said GPR119 agonist is selected from the group consisting of
(2-[((1S)-1-{1-[3-(1-methylethyl)-1,2,4-oxadiazol-5-yl]-4-piperidinyl}ethyl)oxy]-5-[4-(methylsulfonyl)phenyl]pyrazine);
(1-methylethyl 4-({1-[2-fluoro-4-(methylsulfonyl)phenyl]-1H-pyrazolo[3,4-d]pyrimidin-4-yl}oxy)-1-piperidinecarboxylate);
(4-((1R)-3-{[3-fluoro-4-(methylsulfonyl)phenyl]oxy}-1-methylpropyl)-1-[3-(1-methylethyl)-1,2,4-oxadiazol-5-yl]piperidine);
(5-ethyl-2-{4-[4-({[4-(1H-tetrazol-1-yl)phenyl]oxy}methyl)-1,3-thiazol-2-yl]-1-piperidinyl}pyrimidine);
(4-{5-[((1S)-1-{1-[3-(1-methylethyl)-1,2,4-oxadiazol-5-yl]-4-piperidinyl}ethyl)oxy]-2-pyrazinyl}pyridazine);
(1-methylethyl 4-({5-methyl-6-[(2-methyl-3-pyridinyl)oxy]-4-pyrimidinyl}oxy)-1-piperidinecarboxylate hydrochloride);
(1-methylethyl 4-({7-[2-methyl-4-(methylsulfonyl)phenyl]-6,7-dihydro-5H-pyrrolo[2,3-d]pyrimidin-4-yl}oxy)-1-piperidinecarboxylate);
(2-[((1S)-1-{1-[2-(1-methylethyl)-2H-tetrazol-5-yl]-4-piperidinyl}ethyl)oxy]-5-[4-(methylsulfonyl)phenyl]pyrazine);
(1,1-dimethylethyl 4-[((1R)-1-{3-[3-fluoro-4-(methylsulfonyl)phenyl]-1,2,4-oxadiazol-5-yl}ethyl)oxy]-1-piperidinecarboxylate); and pharmaceutically acceptable salts thereof.

6-9. (canceled)
